(19) United States
(12) Patent Application Publication
(10) Pub. No.: US 2025/0268478 A1
(43) Pub. Date: Aug. 28, 2025

(54) Assessment Of Respiratory Depression Risk from a Wearable Device

(71) Applicant: Google LLC, Mountain View, CA (US)

(72) Inventors: Conor Joseph Heneghan, Campbell, CA (US); Alexandros Antonios Pantelopoulos, El Cerrito, CA (US)

(21) Appl. No.: 18/855,259

(22) PCT Filed: Apr. 29, 2022
(86) PCT No.: PCT/US2022/026935

§ 371 Date: Oct. 8, 2024

(51) Int. Cl. A61B 5/0205; A61B 5/00; A61B 5/024; A61B 5/08; A61B 5/091

(52) U.S. Cl. CPC A61B 5/0205; A61B 5/681; A61B 5/7275; A61B 5/02416; A61B 5/02438; A61B 5/0816; A61B 5/091

(57) ABSTRACT

A computing system and method that can be used for determining a respiration rate of the user. In particular, the wearable computing device can determine the respiration rate of the user based at least in part on the heart rate data. Even more particularly, the wearable computing device can determine an overall respiration risk metric. For example, the computing device can compare the overall respiration risk metric to a threshold value (e.g., determined by a machine-learned model or user input). The wearable computing device can determine that the user is at risk for respiratory depression when the overall respiration risk metric satisfies a threshold criteria. Respiratory depression refers to a condition where the drive to breathe is reduced. Specifically, the overall respiration risk metric can represent a likelihood that the user is at risk for respiratory depression.

### FIELD

The present disclosure relates generally to determining a user's respiration rate over time. In particular, the present disclosure is directed to systems and methods for using a respiration risk metric based on data such as heart rate data.

### BACKGROUND

Advances in wearable technology such as fitness trackers and smartwatches have enabled these devices to be used in a variety of contexts. For example, fitness trackers and smartwatches can be used to track a user's heart rate.

### SUMMARY

Aspects and advantages of embodiments of the present disclosure will be set forth in part in the following description, or can be learned from the description, or can be learned through practice of the embodiments.

One example aspect of the present disclosure is directed to a wearable computing device. The wearable computing device includes one or more processors and a heart rate sensor. The wearable computing device further includes a non-transitory computer-readable memory configured to store instructions that cause the one or more processors to perform operations. The operations comprise obtaining via the heart rate sensor heart rate data. The operations comprise determining a respiration rate. The operations comprise determining an overall respiration risk metric associated with a user wearing the wearable computing device, the overall respiration risk metric representing a likelihood that the user is at risk for respiratory depression. The operations comprise determining whether the user is at risk for respiratory depression. The operations comprise providing a notification indicative of the user being at risk for respiratory depression.

Another example aspect of the present disclosure is directed to a computer-implemented method for determining whether a user wearing a wearable computing device is at risk for respiratory depression. The method includes obtaining, via a heart rate sensor, heart rate data. The method includes determining, based at least in part on the hear rate data, a respiration rate. The method includes determining, based at least in part on the respiration rate, an overall respiration risk metric associated with a user wearing the wearable computing device. The overall respiration risk metric represents a likelihood that the user is at risk for respiratory depression. The method includes determining, based at least in part on the overall respiration risk metric, whether the user is at risk for respiratory depression. The method includes providing a notification indicative of the user being at risk for respiratory depression in response to determining the user is at risk for respiratory depression.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

A full and enabling description of the present disclosure, directed to one of ordinary skill in the art, is set forth in the specification, which makes reference to the appended figures, in which:

FIG. 1 depicts a wearable computing device according to some implementations of the present disclosure.

FIG. 2 depicts a block diagram of components of a wearable computing device according to some implementations of the present disclosure.

FIG. 3 depicts an exemplary graphical representation depicting the determined respiration rate over a duration of time according to some implementations of the present disclosure.

FIG. 4 depicts exemplary respiratory effort signals of multiple users according to some implementations of the present disclosure.

FIG. 5 depicts a block diagram demonstrating the one or more metrics taken into account when determining the respiratory depression risk of a user according to some implementations of the present disclosure.

FIG. 6 depicts a flow diagram of a method for determining whether a user is at risk for respiratory depression and providing an indicative notification according to some implementations of the present disclosure.

### DETAILED DESCRIPTION

### Overview

Example aspects of the present disclosure are directed to a wearable computing device that can be worn, for instance, on a user's wrist. The wearable computing device can include one or more processors. The wearable computing device can include a heart rate sensor (e.g., an optical sensor) disposed within a housing of the wearable computing device. The heart rate sensor can determine a heart rate of a user by leveraging photoplethysmography. More particularly, green LED lights (e.g., by flashing them) paired with light-sensitive photodiodes (e.g., to determine green light absorption) can be used to detect the amount of blood flowing through a user's wrist. Additionally, red and infrared photoplethysmography can be used. In this manner, a user's heart rate can be consistently monitored. However, the benefits of such data are limited without additional processing. As such, the wearable computing device determines heart rate, however, since additional processing of the heart rate data is limited, the computing device cannot provide further useful information.

A wearable computing device according to the present disclosure can determine a respiration rate of the user. In particular, the wearable computing device can determine the respiration rate of the user based at least in part on the heart rate data. The wearable computing device can additionally determine whether or not the user has a respiratory sinus arrhythmia based, at least in part, on the heart rate data. Furthermore, the wearable computing device can determine a respiration rate of the user based, at least in part, on the determined respiratory sinus arrhythmia.

In some implementations, the wearable computing device can obtain heart rate data associated with a specific epoch of time. Specifically, the wearable computing device can determine a power spectral density (e.g., based at least in part on the heart rate data associated with the specific epoch of time). The wearable computing device can determine a spectral peak based at least in part on the power spectral density. The respiration rate can be determined based at least in part on the determined spectral peak.

In some implementations, the wearable computing device can determine the respiration rate of the user directly from respiration rate data. For example, the wearable computing device can include a respiration rate sensor configured to obtain respiration data from which the respiration rate of the user can be determined. In alternative implementations, the wearable computing device can determine the respiration rate of the user based on the measured heart rate.

In some implementations, the wearable computing device can determine an overall respiration risk metric. For example, the computing device can compare the overall respiration risk metric to a threshold value (e.g., determined by a machine-learned model or user input). The wearable computing device can determine that the user is at risk for respiratory depression when the overall respiration risk metric satisfies a threshold criteria. Respiratory depression refers to a breathing disorder characterized by slow and ineffective breathing. During a normal breathing cycle, oxygen is inhaled into the lungs. Blood then carries the oxygen around the body and delivering it to various tissues. Blood then takes the carbon dioxide back to the lungs where the carbon dioxide exits the body when a person exhales. However, when a person has respiratory depression, the body cannot adequately remove carbon dioxide which can lead to poor use of oxygen by the lungs. This can result in a higher level of carbon dioxide with too little oxygen available to the body.

In some implementations, the overall respiration risk metric can be based at least in part on the respiration rate of the user wearing the wearable computing device. The overall respiration risk metric can, in some implementations, represent a likelihood that the user is at risk for respiratory depression. As an example, the overall respiration risk metric can include one or more metrics that can be indicative of the user's risk for respiratory depression. For instance, the overall respiration risk metric can be based at least in part on the respiration rate and the one or more metrics. In some implementations, a weighting value can be assigned to the respiration rate and/or the one or more metrics. For instance, the respiration rate and/or the one or more metrics can be provided as an input to a machine-learned model configured to determine a weighted value for the respiration rate and/or the one or more metrics. In alternative implementations, the wearable computing device can assign a predetermined weighting value to the respiration rate and/or the one or more metrics based on a predetermined weighting value. For instance, a linear weighting can be applied. It should be appreciated, however, that any suitable weighting method can be implemented.

In some implementations the one or more metrics associated with the overall respiration risk metric can include a percentage of a predetermined period of time spent with the respiration rate below a threshold value. The threshold value may be predetermined by a machine learned model or, alternatively, by a user. For example, a person whose respiration rate dips below 8 breaths/minute could be considered to have a rate which is too low and should be flagged as high risk for respiratory depression. Thus, the computing device can calculate a percentage of time a user is determined to have a respiration rate below 8 breaths/minute. The one or more metrics can include a number of segments where there is a significant decrease in the respiration rate over a predetermined period of time (e.g., a decrease of >3 breaths per minute in a 5-minute window). The one or more metrics can include one or more percentiles of the respiration rate (e.g., 5th and 10th percentiles). The one or more metrics can include a standard deviation of the respiration rate. The one or more metrics can include an interquartile range of the respiration rate. The one or more metrics can include a kurtosis of a distribution of the respiration rate. The one or more metrics can include a hypoxic event. The one or more metrics can include a bradycardia event.

The wearable computing device can obtain a plurality of demographic factors. For instance, in some implementations, the wearable computing device can obtain a plurality of demographic factors via user input. Specifically, a particular value used in the one or more metrics (e.g., 8 breaths/minute) may be a default threshold which can be adjusted by contextual data. Examples of contextual data can include demographic factors (e.g., age, gender, body mass index, etc.) of the user. In some implementations, the overall respiration risk metric can be determined based, at least in part, on the respiration rate and the contextual data. In some implementations, the wearable computing device can obtain additional contextual data. For example, the additional contextual data can include the user's relevant medical history (e.g., by user input or by user approved input by a medical professional). In some implementations, the relevant medical history can include, but is not limited to, whether the user has sleep apnea or any other neurological breathing issues that may affect the user's respiration rate.

It should be appreciated that the computing system can be configured to safeguard the privacy of personal information about the user. For instance, in some implementations, the computing system can be configured to prompt the user to authorize sharing of contextual data and/or demographic factors provided by the user. For example, the computing system can provide a notification (e.g., text message, email, etc.) to which the user must interact (e.g., reply) with to consent to sharing the contextual data.

In some implementations, the overall respiration risk metric can be based at least in part on at least one of the respiration rate or a determined tidal volume value. In particular, breath-to-breath variability of tidal volume may be ascertained. For example, the wearable computing device can be configured to estimate the tidal volume variability based, at least in part, on a derived respiratory effort signal. The derived respiratory effort signal can be determined by using the green photoplethysmography signal by generating the green photoplethysmography amplitude after baseline removal and creating a time series based on the peak or trough photoplethysmography envelope values. The times series values may increase and decrease with the respiratory effort amplitude so that the tidal volume variability can be estimated by using statistical metrics (e.g., standard deviation of the respiratory effort amplitude over a 1-minute period). In some implementations, the overall respiration risk metric can be based at least in part on at least one of the respiration rates or a determined minute ventilation value. In particular, the determined minute ventilation value can be based on the tidal volume.

In some implementations, the wearable computing device can generate a graphical representation depicting the determined respiration rate over a duration of time. In particular, the wearable computing device can generate the graphical representation based, at least in part, on the determined respiration rate. For instance, the graphical representation can indicate a respiration rate of the user at discrete moments of time or, alternatively, in a continuous period of time. Specifically, an axis of the graphical representation can be adjusted based, at least in part, on the determined respiration rate, the duration of time, or both. For example, the wearable computing device can leverage sliding epochs to generate the graphical representation.

In some implementations, the wearable computing device can generate a graphical representation depicting the determined respiration rate of a user while the user is sleeping. For instance, the wearable computing device can automatically determine that a user is sleeping based, at least in part, on sensor data (e.g., motion data). Alternatively, the wearable computing device can determine that a user is sleeping based at least in part on user input (e.g., a user can indicate that they are in bed).

In some implementations, the wearable computing device can be communicatively coupled to a secondary device via one or more wireless networks. The secondary device can, in some implementations, be an Internet-of-Things (IoT) device. As a particular example, the secondary device can be associated with a smart home. The wearable computing device can interact with the secondary device to select settings associated with the wearable computing device (e.g., thresholds used to determine the overall respiration risk metric). In some implementations, the secondary device may generate relevant data itself. In particular, the secondary device may use non-contact based methods (e.g., using radio frequency sensors) for acquiring data. For instance, in some implementations, the secondary device can be configured to obtain radar data indicative of the motion of the user. The wearable computing device can receive the non-contact based data and determine whether the user risk for respiratory depression based, at least in part, non-contact based data. In some implementations, the determination whether the user is at risk for respiratory depression can be based on the data from the secondary device and data obtained via sensors of the wearable computing device.

The wearable computing device can be configured to provide a notification indicating that the user is at risk for respiratory depression. In particular, the wearable computing device can provide a notification to the user or pre-approved entities that the user is at risk for respiratory depression. For example, the wearable computing device can provide a notification to a user's doctor indicating that the user is at risk for respiratory depression such that the doctor can view the notification and make informed decisions (e.g., about medication dosage) based on the provided information. In some cases, the wearable computing device can provide an auditory (e.g., an alarm), physical (e.g., vibration), or visual notification indicating that the user is at risk for respiratory depression. The visual notification can further include imagery (e.g., the graphical representation depicting the determined respiration rate over time) or words. As another particular example, the wearable computing device can communicate with the connected secondary device such that the secondary computing device provides the notification indicating that the user is at risk for respiratory depression.

In some cases, upon determining that the user is at risk for respiratory depression, the wearable computing device can provide the notification immediately. For example, the wearable computing device can attempt to wake a user from sleep by using a notification (e.g., auditory, vibration) in response to determining that the user is at risk for respiratory depression. In some implementations, the wearable computing device can be communicatively coupled with a secondary device, and the secondary device can attempt to wake a user from sleep as well (e.g., by turning on lights in the user's room, playing music from other connected devices such as speakers, etc.) Specifically, the wearable computing device may provide signals indicating activating a particular wake attempt in a ranked order (e.g., ranked by how jarring the waking method is). Thus, the wearable computing device may attempt to first wake a user by leveraging methods ranked lower before moving up the list if the user does not indicate that they are awake (e.g., by user input to the wearable computing device or to the secondary device). In particular, the wearable computing device may determine whether to move up the list of waking methods one by one or skip particular methods based at least in part on the level of risk. For example, if a user needs to be urgently woken due to a high level of risk the wearable computing device may activate a high ranking method of waking first or quickly jump to a high ranking method rather than attempting lower ranking methods first.

Alternatively, the wearable computing device can compile data associated with the user's risk for respiratory depression and notify the user after a particular amount of time has passed (e.g., after the user has woken up, after the user's exercise has finished, etc.) In particular, the wearable computing device can determine whether to notify the user immediately or after a period of time based on a number of factors (e.g., level of risk, whether the user is engaged in a particular activity such as sleep or exercise, etc.)

A wearable computing device according to example aspects of the present disclosure can provide numerous technical effects and benefits. For instance, by providing a notification to a user indicative of the user being at risk for respiratory depression, the wearable computing device is providing an easy, at-home alternative to cumbersome sleep studies that the user may have otherwise needed to undertake in order to achieve similar results. Alternatively, the user may not have known that they are at risk for respiratory depression and thus the wearable computing device as described in the present disclosure provides an extra safety measure which was not previously available to users.

With reference now to the Figures, example embodiments of the present disclosure will be discussed in further detail.

### Example Devices and Systems

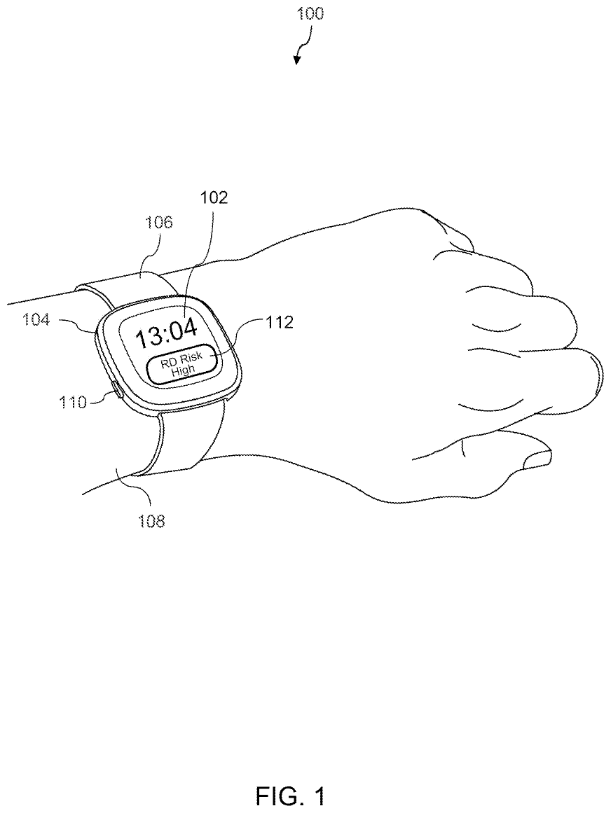

FIG. 1 depicts a wearable computing device 100 according to example embodiments of the present disclosure. As shown, the wearable computing device 100 can be a wristwatch. It should be understood, however, that the wearable computing device 100 can be configured to be worn at other locations on the user's body. For instance, in some implementations, the wearable computing device 100 can be a ring configured to be worn around a user's finger.

In some implementations, the wearable computing device 100 can include a display 102, a device housing 104, a band 106, and one or more sensors. In some implementations, the band 106 can be fastened to an arm 108 of the user to secure the device housing 104 to the arm 108 of the user. In an embodiment, the display 102 can be configured to present to a user data relating to the user's skin temperature, heart rate, sleep state, electroencephalogram, electrocardiogram, electromyography, electrooculogram, and other physiological data of the user (e.g., blood oxygen level). The display 102 can also be configured to convey data from additional ambient sensors contained within the wearable computing device 100. Example information conveyed on the display 102 from these additional ambient sensors can include the positioning, altitude, and weather of a location associated with the user. The display 102 can also convey data regarding the motion of the user (e.g., whether the user is stationary, walking, and/or running).

In some implementations, the display 102 can be configured to display a notification 112. For example, the notification 112 can alert the user that the user is at risk for respiratory depression. The notification 112 can be displayed in real-time when a user is exhibiting symptoms. Alternatively, the notification 112 can be displayed when a user is able to view the notification 112 (e.g., when a user wakes up).

In some implementations, the display 102 can be configured to receive data input by the user. In an embodiment, a user can, by input on the display, request that the wearable computing device 100 generate additional data for display to the user. In response, the display 102 can present instructions to the user to obtain the data. In some examples, the wearable computing device 100 can display instructions to the user (e.g., display “please hold your finger against a sensor for 10 seconds”).

In some implementations, the display 102 can include an interactive display screen (e.g., touchscreen or touch-free screen). In such implementations, the user can interact with the wearable computing device 100 via the display 102 to control operation of the wearable computing device 100. It should be understood that the display 102 can be used to provide content for viewing by the user. For instance, the display 102 can be configured to display a notification indicative of whether the user is at risk for respiratory depression.

In some implementations, the wearable computing device 100 can include one or more input devices 110 that can be manipulated (e.g., pressed) by the user to interact with the wearable computing device 100. For instance, the one or more input devices 110 can include a mechanical button that can be manipulated (e.g., pressed) to interact with the wearable computing device 100. In some implementations, the one or more input devices 110 (e.g., display screen) can be manipulated to control operation of a backlight (not shown) associated with the one or more input devices 110. It should be understood that the one or more input device 110 can be configured to allow the user to interact with the wearable computing device 100 in any suitable manner. For instance, in some implementations, the one or more input devices 110 can be manipulated by the user to navigate through content (e.g., one or more menu screens) displayed on the display 102.

In some implementations, the wearable computing device 100 can include one or more sensors disposed within the device housing 104. For instance, the one or more sensors can include audio sensors, motion sensors (e.g., accelerometer), a pulse oximeter, an IR motion sensor, skin temperature sensors, internal device temperature sensors, location sensors (e.g., GPS), altitude sensors, heart rate sensors, pressure sensors, gyroscopes, environmental sensors (e.g., bedside ultrasounds sensors), and other physiological sensors (e.g., blood oxygen level sensors). In an embodiment, the device housing 104 can also be configured to include one or more processors. The device housing 104 can include a port that connects an audio sensor (e.g., microphone) to the outside of the device housing 104, thus allowing audio information to reach the audio sensor without needing to pass through the device housing 104.

The band 106 can be configured to secure the wearable computing device 100 around an arm 108 of the user by, for example, connecting ends of the band 106 with a buckle, clasp, or another similar securing device, thereby allowing the wearable computing device 100 to be worn by the user.

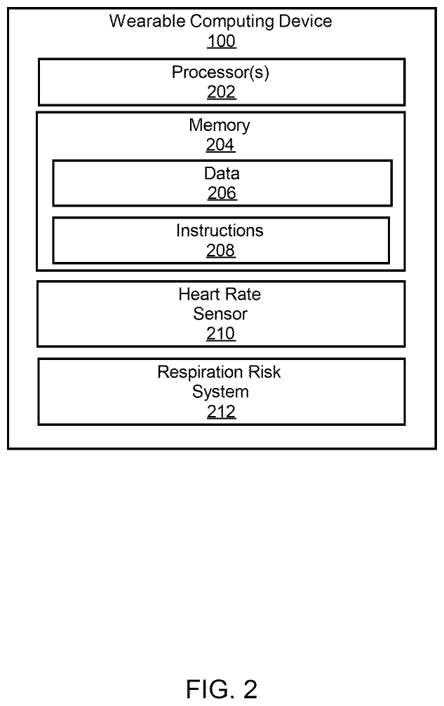

FIG. 2 illustrates an example computing environment including a wearable computing device 100 in accordance with example embodiments of the present disclosure. In this example, the wearable computing device 100 can include one or more processors 202, memory 204, a heart rate sensor 210, and a respiration risk system 212.

In more detail, the one or more processors 202 can be any suitable processing device that can be embedded in the form factor of a wearable computing device 100. For example, such a processor 202 can include one or more of: one or more processor cores, a microprocessor, an application-specific integrated circuit (ASIC), an FPGA, a controller, a microcontroller, etc. The one or more processors 202 can be one processor or a plurality of processors that are operatively connected. The memory 204 can include one or more non-transitory computer-readable storage mediums, such as RAM, ROM, EEPROM, EPROM, flash memory devices, etc., and combinations thereof.

In particular, in some devices, memory 204 can store instructions for implementing the respiration risk system 212. Thus, the wearable computing device 100 can implement a respiration risk system 212 to execute aspects of the present disclosure.

It will be appreciated that the term “system” can refer to specialized hardware, computer logic that executes on a more general processor, or some combination thereof. Thus, a system can be implemented in hardware, application specific circuits, firmware, and/or software controlling a general-purpose processor. In one embodiment, the system can be implemented as program code files stored on the storage device, loaded into memory, and executed by a processor or can be provided from computer program products, such as computer-executable instructions, which are stored in a tangible computer-readable storage medium such as RAM, hard disk or optical or magnetic media.

Memory 204 can also include data 206 and instructions 208 that can be retrieved, manipulated, created, or stored by the one or more processor(s) 202. In some example embodiments, such data can be accessed and used as input to the respiration risk system 212. In some examples, the memory 204 can include data used to perform one or more processes and instructions that describe how those processes can be performed.

In some examples, the heart rate sensor 210 can detect the heart rate associated with the user wearing the wearable computing device 100. For example, the heart rate sensor 210 can determine a heart rate of a user by leveraging photoplethysmography. More particularly, green LED lights (e.g., by flashing them) paired with light-sensitive photodiodes (e.g., to determine green light absorption) can be used to detect the amount of blood flowing through a user's wrist. Additionally, red and infrared photoplethysmography can be used. In this way, the wearable computing device can determine the heart rate of the user.

### Example User Experience

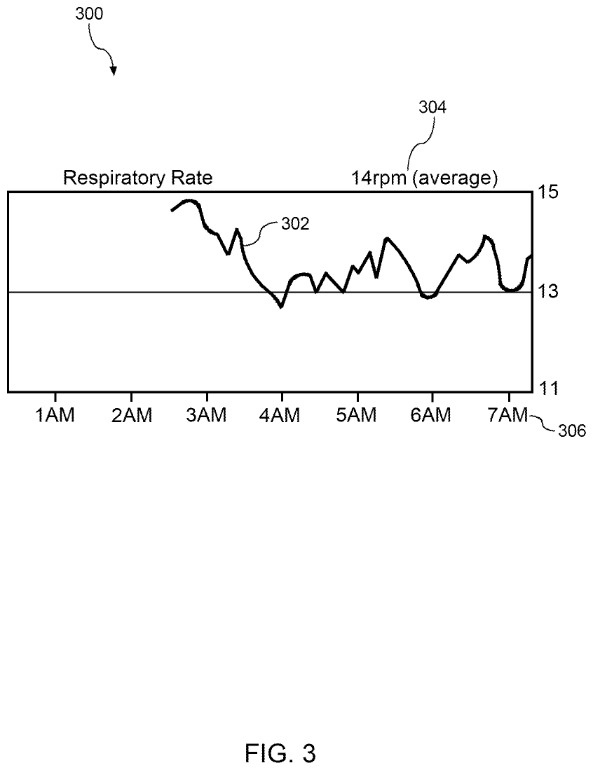

FIG. 3 depicts an example user interface surfacing a graph 300 of a user's respiration rate 302 as a function of time according to some implementations of the present disclosure. For instance, the user respiration rate graph 300 can indicate the user's respiration rate 302 over a time span 306 (e.g., on the x-axis or on the y-axis). In particular, the wearable computing device can determine the user respiration rate 302 based at least in part on heart rate data obtained from the heart rate sensor of the wearable computing device. The wearable computing device can additionally determine respiratory sinus arrhythmia. In particular, the wearable computing device can determine the respiratory sinus arrhythmia based at least in part on the heart rate data.

In some implementations, the wearable computing device can determine the user respiration rate 302 based at least in part on the determined respiratory sinus arrhythmia. For example, the wearable computing device can obtain heart rate data associated with a specific epoch of time. Specifically, the wearable computing device can determine a power spectral density (e.g., based at least in part on the heart rate data associated with the specific epoch of time). The wearable computing device can determine a spectral peak based at least in part on the power spectral density. The user respiration rate 302 can be determined based at least in part on the determined spectral peak.

In some implementations, an average respiration rate 304 can be surfaced alongside the user respiration rate graph 300. In particular, the average respiration rate 304 can be associated with the average respiration rate over the time indicated on the user respiration rate graph 300. Alternatively, the average respiration rate 304 can be associated with respiration rates outside of the time indicated on the graph 300 (e.g., an all-time average respiration rate, a six-month average, a sleeping average, an exercise average, etc.).

In some implementations, the graph 300 can surface the user respiration rate 302 for alternative time spans 306. For example, the user respiration rate graph 300 can surface user respiration rates 302 for different time spans 306 based at least in part on user interaction indicating a user desire to see a different time span 306 (e.g., past year, past month, past day, etc.).

In some implementations, the graph 300 can indicate when the user respiration rate 302 is below or above a threshold value. The threshold value may be predetermined by a machine learned model or, alternatively, by a user. For example, a user respiration rate 302 below 8 breaths/minute could be considered to have a rate which is too low and should be flagged as high risk for respiratory depression. A In some implementations, the graph 300 can surface icons indicating that the user respiration rate 302 is high risk. As another particular example, the user respiration rate graph 300 can change the color of the user respiration rate 302 line graph indicating the high-risk data.

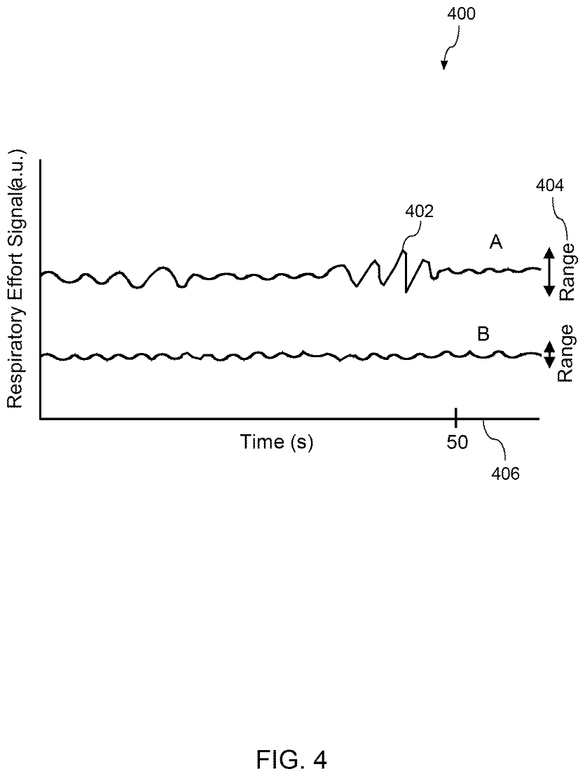

FIG. 4 depicts an example user interface surfacing a user respiratory effort signal graph 400. In particular, the user respiratory effort signal graph 400 can display a user respiratory effort signal 402 over time according to an example embodiment of the present disclosure. Specifically, the user respiratory effort signal graph 400 can indicate a time span 404 (e.g., on the x-axis or on the y-axis). Specifically, the respiratory effort signal 402 can be determined by using a green photoplethysmography signal by generating a green photoplethysmography amplitude after baseline removal and creating a time series based on the peak or trough photoplethysmography envelope values. The times series values may increase and decrease with the respiratory effort amplitude so that a tidal volume variability can be estimated by using statistical metrics (e.g., standard deviation of the respiratory effort amplitude over a 1-minute period).

In some implementations, a time span 404 of respiratory effort signal 402 can be surfaced to the user. In particular, the time span 404 of the respiratory effort signal 402 over a particular time span 406 can be determined and surfaced for the user.

In some implementations, the user respiratory effort signal graph 400 can surface the respiratory effort signal 402 for alternative time spans 406. For example, the user respiratory effort signal graph 400 can surface respiratory effort signals 402 for alternative time spans 406 based at least in part on user interaction indicating a user desire to see an alternative time span 406 (e.g., last night of sleep, last exercise, etc.).

### Example Models

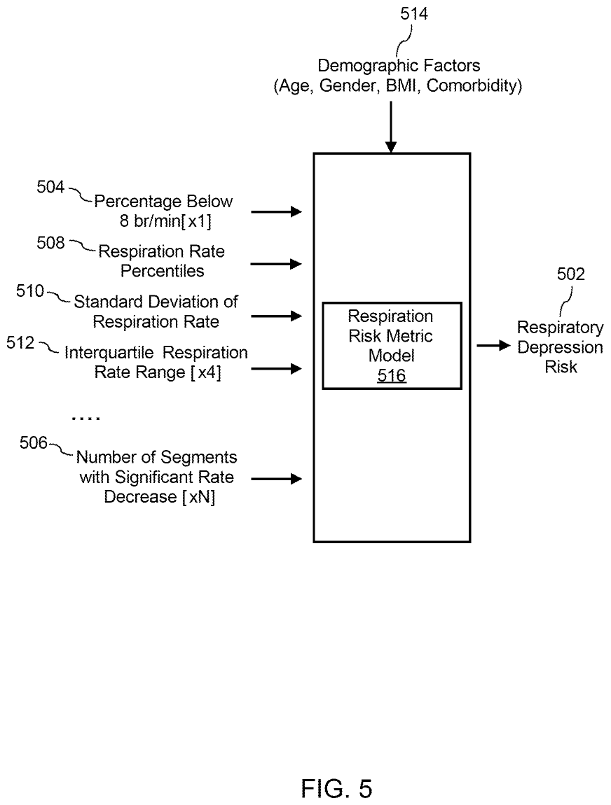

FIG. 5 depicts an example model predicting a respiratory depression risk of a user associated with a wearable computing device (e.g., wearable computing device 100 with reference to FIG. 1). In particular, the computing device can compare the overall respiration risk metric 502 to a threshold value (e.g., determined by a machine-learned model or user input). The wearable computing device can determine that the user is at risk for respiratory depression when the overall respiration risk metric 502 satisfies a threshold criteria. In particular, the overall respiration risk metric 502 can be based at least in part on the respiration rate. Even more particularly, the overall respiration risk metric 502 can be associated with a user wearing the wearable computing device. Specifically, the overall respiration risk metric 502 can represent a likelihood that the user is at risk for respiratory depression. As an example, the respiration risk metric 502 can include one or more metrics associated with the risk for respiratory depression. For instance, the overall respiration risk metric 502 can be predicted based at least in part on an input respiration rate and one or more metrics.

In some implementations, the overall respiration risk metric 502 can be predicted based on a machine-learned model (e.g., a respiration risk metric model 516). In particular, the respiration rate and the one or more metrics can be input into the respiration risk metric model 516. The respiration risk metric model 516 can leverage the input respiration rate and one or more metrics to output an overall respiration risk metric 502.

In some implementations, the respiration rate and the one or more metrics can be weighted by the wearable computing device. Specifically, the wearable computing device can weight the respiration rate and the one or more metrics based on a machine-learned model generating weighting values. In some implementations, the wearable computing device can weight the respiration rate and the one or more metrics based on a predetermined weighting value. For instance, a linear weighting, or any other appropriate weighting method may be used.

For instance, the one or more metrics can include a first input 504 (i.e., to the respiration risk metric model), a percentage of a predetermined period of time spent with the respiration rate below a threshold value. The computing device can calculate a percentage of time a user is determined to have a respiration rate below 8 breaths/minute to determine the first input 504. The one or more metrics can include a second input 506. The second input 506 refers to a calculated number of segments where there is a significant decrease in the respiration rate over a predetermined period of time (e.g., a decrease of >3 breaths per minute in a 5-minute window). The one or more metrics can include a third input 508. The third input refers to one or more percentiles of the respiration rate (e.g., 5th and 10th percentiles). The one or more metrics can include a fourth input 510. The fourth input 510 refers to a standard deviation of the respiration rate. The one or more metrics can include a fifth input 512. The fifth input 512 refers to an interquartile range of the respiration rate. Inputs are not limited to those shown in FIG. 5. For example, the one or more metrics can include the heart rate variability (HRV) of the user. In particular, a heart rate sensor (e.g., heart rate sensor 210 of FIG. 2) may be configured to calculate the interbeat intervals between each cardiac beat (e.g., at a heart rate of 60 bpm, there is typically an approximate 1 second interval between each beat). From this time set of intervals, HRV metrics can be obtained. For example, a set of HRV metrics can be calculated over the last five minutes of the recording. Typical HRV metrics would include (a) the average interbeat interval, (b) the root mean square of successive differences (RMSSD), (c) the pNN50, which is the percentage of successive interbeat intervals that differ by more than 50 ms, (d) the low frequency (LF) spectral power of the interbeat interval spectrum, and (e) the high frequency (HF) spectral power of the interbeat interval a kurtosis of a distribution of the respiration rate. As another example, the one or more metrics can include derivations from red and infrared photoplethysmography associated with the wearable computing device. In particular, the system can determine the estimated SpO2 using known techniques in oximetry (such as the “ratio of ratios”) Respiratory depression risk can then be obtained by looking for periods where the estimated SpO2 falls below a threshold (e.g., 92% for prolonged periods of time) or where there is excessive variability in the baseline of the estimated SpO2. Further examples of metrics include a hypoxic event and a bradycardia event.

The wearable computing device can obtain a plurality of demographic factors 514. For instance, in some implementations, the wearable computing device can obtain a plurality of demographic factors 514 via user input. Specifically, a particular value used in the one or more metrics (e.g., 8 breaths/minute) may be a default threshold which can be adjusted by contextual data. Examples of contextual data can include demographic factors 514 (e.g., age, gender, body mass index, etc.) of the user. In some implementations, the overall respiration risk metric 502 can be determined based, at least in part, on the respiration rate and the contextual data. In some implementations, the wearable computing device can obtain additional contextual data. For example, the additional contextual data can include the user's relevant medical history (e.g., by user input or by user approved input by a medical professional). In some implementations, the relevant medical history can include, but is not limited to, whether the user has sleep apnea or any other neurological breathing issues that may affect the user's respiration rate.

In some implementations, the overall respiration risk metric 502 can be based at least in part on at least one of the respiration rate or a determined tidal volume value. In particular, breath-to-breath variability of tidal volume may be ascertained. For example, the wearable computing device can be configured to estimate the tidal volume variability based, at least in part, on a derived respiratory effort signal. In some implementations, the overall respiration risk metric can be based at least in part on at least one of the respiration rates or a determined minute ventilation value. In particular, the determined minute ventilation value can be based on the tidal volume.

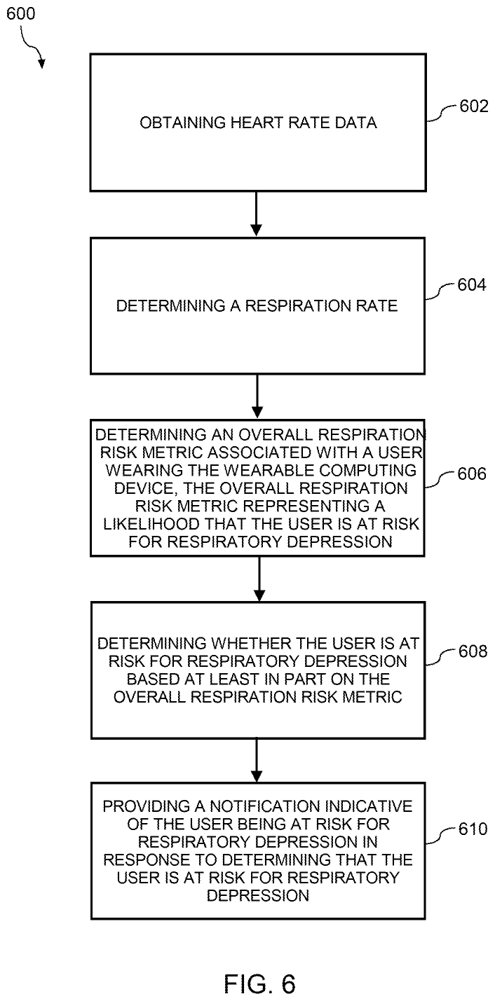

FIG. 6 depicts a flow chart diagram of an example method to perform according to example embodiments of the present disclosure. Although FIG. 6 depicts steps performed in a particular order for purposes of illustration and discussion, the methods of the present disclosure are not limited to the particularly illustrated order or arrangement. The various steps of the method 600 can be omitted, rearranged, combined, and/or adapted in various ways without deviating from the scope of the present disclosure. In particular, the example method depicted in FIG. 6 may be performed entirely by the wearable computing device. Alternatively, in some implementations, the example method depicted in FIG. 6 may be performed by multiple devices such that the example method is split between the multiple devices. In particular, the multiple devices can include the wearable computing device and a remote computing device (e.g., a server).

At 602, a computing system comprising one or more computing devices can obtain heart rate data. In particular, the computing system can obtain the heart rate data from a heart rate sensor. The wearable computing device can additionally determine respiratory sinus arrhythmia. In particular, the wearable computing device can determine the respiratory sinus arrhythmia based at least in part on the heart rate data. Furthermore, the wearable computing device can determine the respiration rate of the user based at least in part on the determined respiratory sinus arrhythmia. As a particular example, the wearable computing device can obtain heart rate data associated with a specific epoch of time. Specifically, the wearable computing device can determine a power spectral density (e.g., based at least in part on the heart rate data associated with the specific epoch of time). The wearable computing device can determine a spectral peak based at least in part on the power spectral density.

At 604, the computing system can determine a respiration rate. In particular, the computing system can determine the respiration rate based at least in part on the heart rate data (e.g., the heart rate data obtained from the heart rate sensor). The respiration rate can be determined based at least in part on the determined spectral peak.

At 606, the computing system can determine an overall respiration risk metric. In particular, the overall respiration risk metric may be associated with a user wearing the wearable computing device. Even more particularly, the overall respiration risk metric can represent a likelihood that the user may be at risk for respiratory depression. Specifically, the overall respiration risk metric can be based at least in part on the respiration rate (e.g., the determined respiration rate, a measured respiration rate, etc.). For example, the computing device can compare the overall respiration risk metric to a threshold value. In particular, the overall respiration risk metric can be predicted by a machine-learned model based on an input respiration rate and one or more metrics. Furthermore, the threshold value may be additionally predicted by a machine-learned model. The threshold value may be predicted by a machine-learned model based at least in part on demographic factors or external standards which can be input by a manufacturer or by the user.

At 608, the computing system can determine whether the user is at risk for respiratory depression. In particular, the computing system can determine whether the user is at risk for respiratory depression based at least in part on the overall respiration risk metric. The wearable computing device can determine that the user is at risk for respiratory depression when the overall respiration risk metric satisfies a threshold criteria. In particular, the overall respiration risk metric can be based at least in part on the respiration rate. Even more particularly, the overall respiration risk metric can be associated with a user wearing the wearable computing device. Specifically, the overall respiration risk metric can represent a likelihood that the user is at risk for respiratory depression.

At 610, the computing system can provide a notification indicative of the user being at risk for respiratory depression. In particular the computing system can provide the notification indicative of the use being at risk for respiratory depression in response to determining the user is at risk for respiratory depression. In particular, the wearable computing device can provide a notification to the user or pre-approved entities that the user is at risk for respiratory depression. For example, the wearable computing device can provide a notification to a user's doctor indicating that the user is at risk for respiratory depression such that the doctor can view the notification and make informed decisions (e.g., about medication dosage) based on the provided information. In some cases, the wearable computing device can provide an auditory (e.g., an alarm), physical (e.g., vibration), or visual notification indicating that the user is at risk for respiratory depression. The visual notification can further include imagery (e.g., the graphical representation depicting the determined respiration rate over time) or words. As another particular example, the wearable computing device can communicate with the connected secondary device such that the secondary computing device provides the notification indicating that the user is at risk for respiratory depression.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A wearable computing device, comprising:
one or more processors,
a heart rate sensor;
a non-transitory computer-readable memory, the non-transitory computer-readable memory configured to store instructions that, when executed by the one or more processors, cause the one or more processors to perform operations, the operations comprising:
obtaining, via the heart rate sensor, heart rate data;
determining, based at least in part on the heart rate data, a respiration rate;
determining, based at least in part on the respiration rate, an overall respiration risk metric associated with a user wearing the wearable computing device, the overall respiration risk metric representing a likelihood that the user is at risk for respiratory depression;
determining, based at least in part on the overall respiration risk metric, whether the user is at risk for respiratory depression; and
responsive to determining the user is at risk for respiratory depression, providing a notification indicative of the user being at risk for respiratory depression.

2. The wearable computing device of claim 1, wherein determining whether the user is at risk for respiratory depression comprises:
comparing the overall respiration risk metric to a threshold value; and
determining the user is at risk for respiratory depression when the overall respiration risk metric satisfies a threshold criteria.

3. The wearable computing device of claim 1, wherein the heart rate sensor comprises one or more optical sensors.

4. The wearable computing device of claim 1, wherein determining the respiration rate comprises:
determining respiratory sinus arrhythmia based at least in part on the heart rate data; and
determining, based at least in part on the respiratory sinus arrhythmia, the respiration rate.

5. The wearable computing device of claim 4, wherein determining the respiration rate further comprises:
obtaining, via the heart rate sensor, heart rate data associated with a specific epoch of time;
determining, based at least in part on the heart rate data associated with the specific epoch of time, a power spectral density;
determining, based at least in part on the power spectral density, a spectral peak; and
determining, based at least in part on the spectral peak, the respiration rate.

6. The wearable computing device of claim 1, wherein the operations further comprising:
generating, based at least in part on the determined respiration rate, a graphical representation depicting the determined respiration rate over a duration of time.

7. The wearable computing device of claim 1, wherein determining the overall respiration risk metric comprises:
determining, based at least in part on the respiration rate, at least one metric associated with the risk for respiratory depression, wherein the at least one metric comprises:
a percentage of a predetermined period of time spent with the respiration rate below a threshold value;
determining the overall respiration risk metric based, at least in part, on the respiration rate and the one or more metrics.

8. The wearable computing device of claim 1, wherein determining the overall respiration risk metric comprises:
obtaining, via user input, a plurality of demographic factors;
determining the overall respiration risk metric based at least in part on at least one of the respiration rate, or one or more of the plurality of demographic factors.

9. The wearable computing device of claim 1, wherein determining the overall respiration risk metric is based, at least in part, on at least one of the respiration rate or a determined minute ventilation value.

10. The wearable computing device of claim 1, wherein determining the overall respiration risk metric is based, at least in part, on at least one of the respiration rate or a determined tidal volume value.

11. The wearable computing device of claim 10, wherein the overall respiration risk metric is based, at least in part, on at least one of the respiration rate or a tidal volume variability value that is based at least in part on the determined tidal volume value.

12. A computer-implemented method for determining whether a user wearing a wearable computing device is at risk of respiratory depression, the method comprising:
obtaining, via a heart rate sensor, heart rate data;
determining, based at least in part on the heart rate data, a respiration rate;
determining, based at least in part on the respiration rate, an overall respiration risk metric associated with a user wearing the wearable computing device, the overall respiration risk metric representing a likelihood that the user is at risk for respiratory depression;
determining, based at least in part on the overall respiration risk metric, whether the user is at risk for respiratory depression; and
responsive to determining the user is at risk for respiratory depression, providing a notification indicative of the user being at risk for respiratory depression.

13. The computer-implemented method of claim 12, wherein determining whether the user is at risk for respiratory depression comprises:
comparing the overall respiration risk metric to a threshold value; and
determining the user is at risk for respiratory depression when the overall respiration risk metric satisfies a threshold criteria.

14. The computer-implemented method of claim 12, wherein the heart rate sensor comprises one or more optical sensors.

15. The computer-implemented method of claim 12, further comprising:
generating, based at least in part on the determined respiration rate, a graphical representation of the determined respiration rate over a duration of time.

16. The computer-implemented method of claim 12, wherein determining the overall respiration risk metric comprises:
determining, based at least in part on the respiration rate, at least one metric associated with the risk for respiratory depression, wherein the at least one metric comprises a percentage of a predetermined period of time spent with the respiration rate below a threshold value; and
determining the overall respiration risk metric based, at least in part, on the respiration rate and the at least one metric.

17. The computer-implemented method of claim 12, wherein determining the overall respiration risk metric comprises:
obtaining, via user input, a plurality of demographic factors;
determining, based at least in part on a combination of the respiration rate and the plurality of demographic factors, the overall respiration risk metric.

18. The computer-implemented method of claim 12, wherein determining the overall respiration risk metric is based at least in part on a combination of the respiration rate and a determined minute ventilation value.

19. The computer-implemented method of claim 12, wherein determining the overall respiration risk metric is based at least in part on a combination of the respiration rate and a determined tidal volume value.

20. The computer-implemented method of claim 19, wherein the overall respiration risk metric is based at least in part on a combination of the respiration rate and a tidal volume variability value based at least in part on the determined tidal volume value.
